(19) United States
(12) Patent Application Publication
(10) Pub. No.: US 2017/0311991 A1
(43) Pub. Date: Nov. 2, 2017

(54) INTERSPINOUS INTEGRATION TYPE IMPLANT

(71) Applicants: Hyeun-Sung Kim, Daejeon (KR); SOLCO BIOMEDICAL CO., LTD., Pyeongtaek-si (KR)

(72) Inventors: Hyeun-Sung Kim, Daejeon (KR); Hong-Won Yoon, Gunpo-si (KR)

(21) Appl. No.: 15/520,166

(22) PCT Filed: Oct. 19, 2015
(86) PCT No.: PCT/KR2015/011009

§ 371 Date: Apr. 19, 2017

(30) Foreign Application Priority Data
Oct. 20, 2014 (KR) 10-2014-0142122

(51) Int. Cl. A61B 17/70

(52) U.S. Cl. CPC A61B 17/7067; A61B 17/7068; A61B 17/7061

(57) ABSTRACT

The present invention relates to an interspinous integration type implant. Provided is an interspinous integration type implant which is an implant inserted between an upper spinous process and a lower spinous process and comprises: a spacer to which one end of an upper plate and one end of a lower plate are connected so as to be formed in a “ [symbol] ” shape or “U” shape, and which is inserted between spinous processes in order to provide elasticity; an upper spinous process coupling means for tightly coupling the upper plate to the upper spinous process; and a lower spinous process coupling means for tightly coupling the lower plate to the lower spinous process, wherein the upper plate comprises at least one osseointegration hole formed to penetrate the top surface and the bottom surface of the upper plate so as to be moved by being integrated with the upper spinous process, and the lower plate comprises at least one osseointegration hole formed to penetrate the top surface and the bottom surface of the lower plate so as to be moved by being integrated with the lower spinous process. The interspinous integration type implant according to the present invention provides elasticity to the upper spinous process and the lower spinous process, thereby maintaining a space between the upper and lower spinous processes. Also, the interspinous integration type implant moves by being integrated with the upper and lower spinous processes through the osseointegration holes formed at the upper plate and the lower plate, and thus does not damage the spinous processes and has an effect of preventing a separation phenomenon from between the spinous processes. Also, the interspinous integration type implant has an effect of enabling minimally invasive surgery since blades can be rotated and tightened using an instrument after inserting the interspinous integration type implant from the side surface of spinous processes. Furthermore, after inserting the interspinous integration type implant between adjacent spinous processes and tightening the blades at both side surfaces of the spinous processes, a plurality of spikes provided in the blades are tightly embedded on the both side surfaces of the spinous processes, and thus there is an effect that an upper blade and a lower blade are stably coupled to the upper and lower spinous processes. In addition, since the interspinous integration type implant can be inserted between the spinous processes in a state where the upper blade and the lower blade are folded, the blades do not catch on the upper and lower spinous processes during insertion, and thus there is an effect that the interspinous integration type implant can be inserted not only from the rear side of the spinous processes but also from the side surface of the spinous processes.

### BACKGROUND OF THE INVENTION

### Field of the invention

The present invention relates to an interspinous fusion type implant and, more particularly, to an interspinous fusion type implant inserted between adjacent spinous processes in order to treat spinal canal stenosis which is generated because a spinal canal, nerve root canal or intervertebral foramen at the center of a spine is narrowed.

### Related Art

A spinal canal is a passage through which a vertebral nerve that is connected to the brain of the head, that transfers a signal generated by the brain to an arm and leg, and that functions as an “electric wire” passes. Holes at the rear of respective vertebrae are connected up and down to form a tunnel, and nerves continue from the brain to the arm and leg through the tunnel. Spinal canal stenosis is a disease in which a passage through which such a vertebral nerve passes is narrowed to presses the nerve. The most commonly generated degenerative stenosis is generated because as a vertebra experiences an aging process, a disc (intervertebral disc) that has caused a degenerative change at the front pops out and presses a nerve, a ligamentum flava that surrounds the nerve becomes thick and hard-set to press the nerve at the back, a facet joint located at the back of a spinal canal is swollen, and a vertebral nerve is pressed by a bone (osteophyte) abnormally grown by the friction of the vertebra.

Recently, in order to treat such spinal canal stenosis, an implant inserted between adjacent spinous processes is widely used. However, a conventional implant has problems in that a spinous process is damaged because upper and lower spinous processes are not fused and are separately moved and thus a vertebra moves for a long period of time and also the implant escapes between the spinous processes.

Furthermore, there are problems in that a long operation time is taken because a ligament must be cut in the rear of the back and the implant must be inserted in order to insert the implant between the spinous processes, and the time taken for a patient to recover after the operation is long.

Furthermore, there is a problem in that the implant is not coupled to the upper and lower spinous processes perfectly and stably because a wing portion coupled to the upper and lower spinous processes is not configured to be adjusted according to the thickness of the spinous process after the implant is inserted between the spinous processes.

There are Korean Patent No. 10-1346095 and Korean Patent Application Publication No. 10-2010-0080908 as prior art documents.

### SUMMARY OF THE INVENTION

The present invention has been invented to improve the problems, and an object of the present invention is to provide an interspinous process fusion type implant, which can maintain the space between an upper spinous process and a lower spinous process because the implant is inserted between adjacent spinous processes to provide an elastic force to the upper spinous process and the lower spinous process and which can be integrated with the upper and lower spinous processes and moved therewith.

Furthermore, an object of the present invention is to provide an interspinous fusion type implant capable of a minimum invasion procedure because wings are rotated and fastened using a tool after the implant is inserted on the side of spinous processes without using a method of inserting the implant at the rear of the spinous process.

Furthermore, an object of the present invention is to provide an interspinous fusion type implant in which an upper wing and a lower wing are coupled to a spinous process perfectly and stably by fastening the wings on both sides of the spinous process after the implant is inserted between adjacent spinous processes.

An interspinous fusion type implant of the present invention for achieving the objects is an interspinous fusion type implant inserted between an upper spinous process and a lower spinous process, the implant including a spacer formed in “ [symbol] ” shape or “U” shape and inserted between spinous processes to provide an elastic force, one ends of an upper plate and a lower plate being connected to the spacer; upper spinous process coupling means which closely couples the upper plate to an upper spinous process; and lower spinous process coupling means which closely couples the lower plate to a lower spinous process. The upper plate includes one or more osseointegration holes formed to penetrate the upper plate from the top surface of the upper plate to the bottom surface of the upper plate in such a way as to be fused with the upper spinous process and to move. The lower plate includes one or more osseointegration holes formed to penetrate the lower plate from the top surface of the lower plate to the bottom surface of the lower plate in such a way as to be fused with the lower spinous process and to move.

Furthermore, a plurality of fixing protrusions may be formed in the top surface of the upper plate which belongs to the upper plate and in which the osseointegration hole has not been formed, and may be coupled to the upper spinous process. A plurality of fixing protrusions may be formed in the bottom surface of the lower plate which belongs to the lower plate and in which the osseointegration hole has not been formed, and may be coupled to the lower spinous process.

Furthermore, a side portion of the upper plate corresponding to a portion in which the osseointegration hole is formed may further include an osseointegration hole formed to penetrate the side portion of the upper plate in such a way as to be connected to the osseointegration hole. A side portion of the lower plate corresponding to a portion in which the osseointegration hole is formed may further include an osseointegration hole formed to penetrate the side portion of the lower plate in such a way as to be connected to the osseointegration hole.

Furthermore, the upper spinous process coupling means may be upward rotated by a first rotation rod inserted into a through hole formed to penetrate the side of the upper plate and rotated with the upper plate, closely attached to both sides of the upper spinous process, and coupled to the upper spinous process. The lower spinous process coupling means may be downward rotated by a second rotation rod inserted into a through hole formed to penetrate the side of the lower plate and rotated with the lower plate, closely attached to both sides of the lower spinous process, and coupled to the lower spinous process.

Furthermore, an upper wing coupled to the first rotation rod through the first rotation rod may be upward rotated. A lower wing coupled to the second rotation rod through the second rotation rod may be downward rotated. The upper wing and the lower wing may be closely attached and mutually fastened on the first rotation rod and the second rotation rod, respectively, so a plurality of spinous process fixing spikes provided in the upper wing and a plurality of spinous process fixing spikes provided in the lower wing are embedded in the upper spinous process and the lower spinous process.

Furthermore, the through hole formed to penetrate the side of the upper plate and the through hole formed to penetrate the side of the lower plate may be formed at mutually dislocated locations so that the first rotation rod is provided more on a front side than the second rotation rod. The upper wing coupled to the first rotation rod may be upward rotated and the lower wing coupled to the second rotation rod may be downward rotated by rotating the first rotation rod and the second rotation rod in an identical direction.

Furthermore, oblong through holes to which the first rotation rod and the second rotation rod are coupled through the oblong through holes may be formed on the lower sides of the upper wing and the lower wing, respectively. A plurality of spinous process fixing spikes may be provided on the upper side of each of the upper wing and the lower wing. The outer surfaces of the first rotation rod and the second rotation rod may be formed in shapes capable of rotating the upper wing and the lower wing coupled to the first rotation rod and the second rotation rod through the first rotation rod and the second rotation rod.

Furthermore, the first rotation rod and the second rotation rod may have bolt forms. A bolt head may be located on one side of each of the upper plate and the lower plate. A bolt body may be exposed to the other side through the through hole. A left wing forming the upper wing may be coupled to a bolt body exposed between the bolt head and the upper plate. A left wing forming the lower wing may be coupled to a bolt body exposed between the bolt head and the lower plate. The right wing forming the upper wing and the right wing forming the lower wing may be coupled to the bolt body exposed to the other side.

Furthermore, the outer surfaces of the bolt bodies corresponding to portions coupled to the oblong through holes formed in the left wing and the right wing may include a plurality of faces flat cut in such a way as to rotate the left and right wings when the bolt bodies are rotated. Furthermore, a fastening nut may be coupled to an outer bolt body of the right wing. When the fastening nut is fastened, the right wing may move in the direction of the left wing along the bolt body and the left wing may move in the direction of the right wing along the bolt body, so the left wing and the right wing are closely attached and fastened in a direction in which the left wing and the right wing face each other.

Furthermore, after the left and right wings are closely attached and fastened by the fastening nut, a fixing nut may be coupled to the outer bolt body of the fastening nut.

In accordance with the interspinous fusion type implant according to the present invention, there are advantages in that the space between the upper spinous process and the lower spinous process can be maintained because an elastic force is provided to the upper spinous process and the lower spinous process and a phenomenon in which the interspinous fusion type implant escapes between the upper spinous process and the lower spinous process can be prevented without damaging the spinous process because the implant is fused with the upper and lower spinous processes and moved therewith through the osseointegration holes formed in the upper plate and the lower plate.

Furthermore, there is an advantage in that a minimum invasion procedure is possible because the wings are rotated and fastened using a tool after the interspinous fusion type implant is inserted from the sides of the spinous processes.

Furthermore, there is an advantage in that the upper wing and the lower wing are stably coupled to the upper and lower spinous processes because the plurality of spikes included in the upper and lower wings are closely attached to both sides of the upper and lower spinous processes and embedded therein when the upper and lower wings are fastened on both sides of the upper and lower spinous processes after the interspinous fusion type implant is inserted between the adjacent spinous processes.

Furthermore, there is an advantage in that the interspinous fusion type implant can be inserted from the sides of the spinous processes as well as at the back of the spinous processes because the implant can be inserted between the spinous processes in the state in which the upper wing and the lower wing have been folded and thus the wings are not caught in the upper and lower spinous processes.

### BRIEF DESCRIPTION OF THE DRAWINGS

FIG. 1 is a side view showing the state in which an interspinous fusion type implant according to the present invention has been inserted between spinous processes.

FIG. 2 is a perspective view showing the state in which the wings of the interspinous fusion type implant according to the present invention have been spread and then finally fixed.

FIG. 3 is a side view showing the state in which the wings of the interspinous fusion type implant according to the present invention have been spread and then finally fixed.

FIG. 4 is a plan view showing the state in which the wings of the interspinous fusion type implant according to the present invention have been spread and then finally fixed.

FIG. 5 is a perspective view showing the state when the interspinous fusion type implant according to the present invention is inserted between spinous processes after the wings of the implant is folded.

FIG. 6 is a perspective view showing the state in which the wings have been spread after the interspinous fusion type implant according to the present invention was inserted between spinous processes.

FIG. 7 is a plan view showing the state in which the wings have been spread after the interspinous fusion type implant according to the present invention was inserted between spinous processes.

FIG. 8 is a front view showing the state in which the wings have been spread after the interspinous fusion type implant according to the present invention was inserted between spinous processes.

FIG. 9 is a front view showing the state in which left and right wings have been fastened by fastening a fastening nut after the interspinous fusion type implant according to the present invention was inserted between spinous processes.

FIG. 10 is a front view showing the state in which the left and right wings of the interspinous fusion type implant according to the present invention have been fixed using a fixing nut after the left and right wings were fastened.

FIG. 11 is a side view of a spacer inserted between spinous processes.

FIG. 12 is a perspective view of a rotation rod which is inserted into a through hole formed on the side of the spacer and rotated.

FIG. 13 is a perspective view of the wing including a plurality of spinous process fixing spikes.

### DESCRIPTION OF EXEMPLARY EMBODIMENTS

Hereinafter, an embodiment according to the present invention is described in detail with reference to the accompanying drawings.

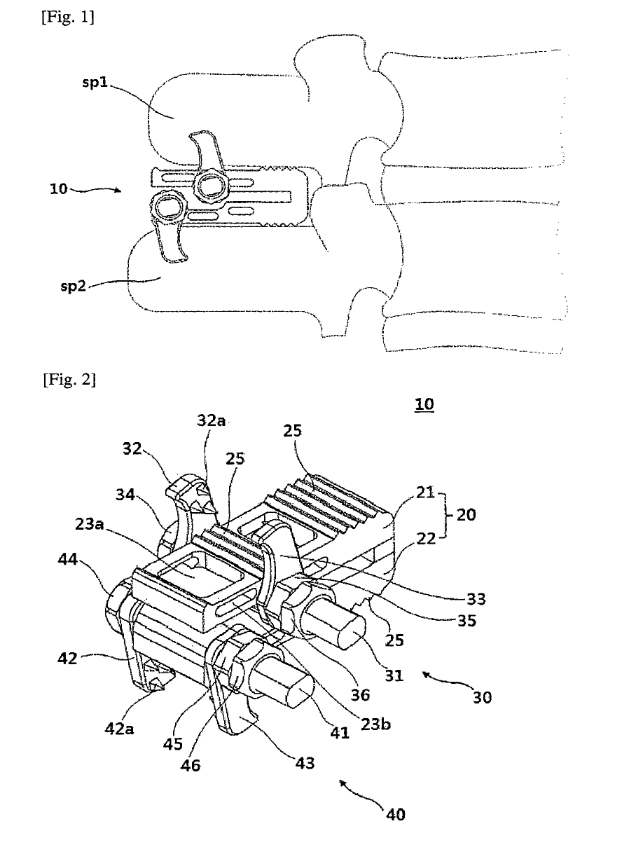

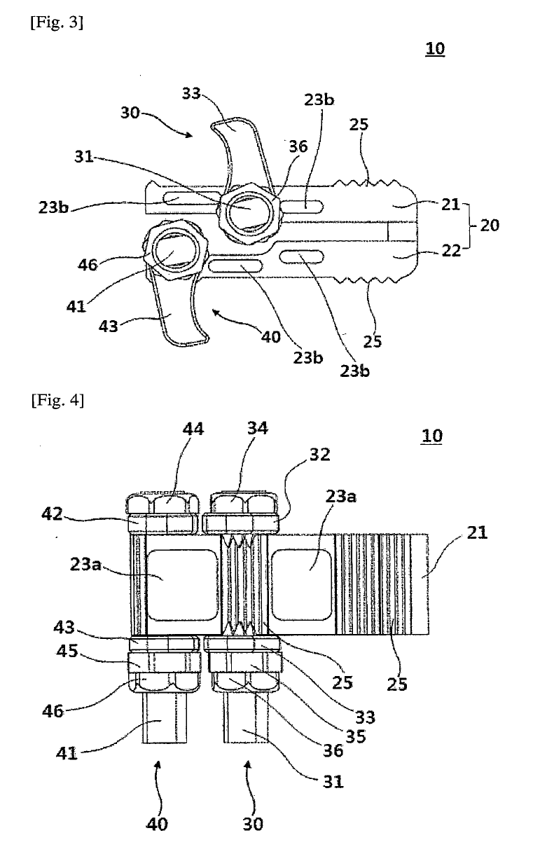

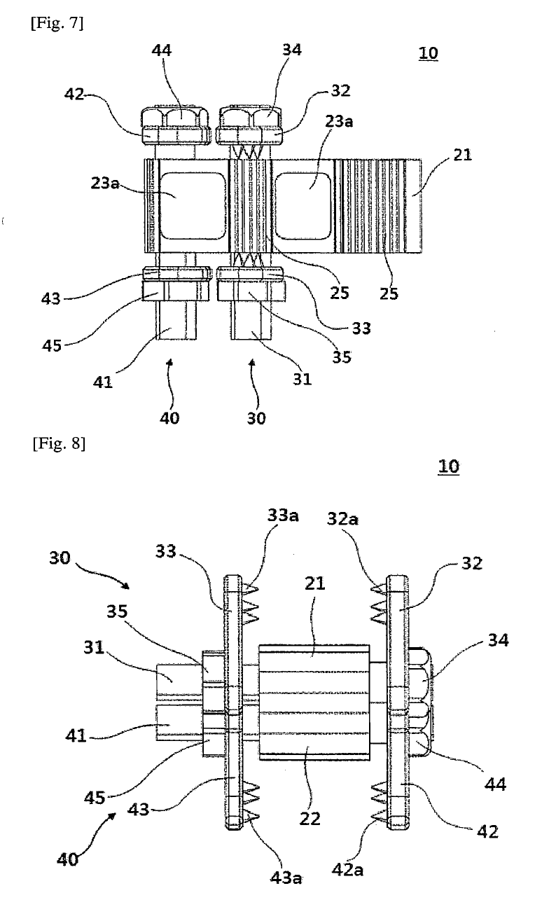

Referring to FIGS. 1 to 7, an interspinous integration type implant (10) includes a spacer (20) inserted between spinous processes (sp1 and sp2), upper spinous process coupling means (30), and lower spinous process coupling means (40).

The spacer (20) is connected to one ends of an upper plate (21) and a lower plate (22) to form a ‘ [symbol] ’ shape or a ‘U’ shape. The spacer (20) provides an elastic force to the adjacent spinous processes (sp1 and sp2).

The upper spinous process coupling means 30 closely couples the upper plate (21) to the upper spinous process (sp1), and the lower spinous process coupling means (40) closely couples the lower plate (22) to the lower spinous process (sp2).

The upper plate (21) includes one or more osseointegration holes (23a) formed to penetrate the upper plate (21) from the top surface of the upper plate (21) to the bottom surface thereof so that the upper plate (21) is fused with the upper spinous process (sp1) and moved therewith. The lower plate (22) includes one or more osseointegration holes (23a) formed to penetrate the lower plate (22) from the top surface of the lower plate (22) to the bottom surface thereof so that the lower plate (22) is fused with the lower spinous process (sp2) and moved therewith. When the osseointegration hole (23a) is filled with bone powder, the bone powder filled in the osseointegration hole (23a) formed in the upper plate (21) is fused with the upper spinous process (sp1). The bone powder filled in the osseointegration hole (23b) formed in the lower plate (22) is fused with the lower spinous process (sp2).

A plurality of fixing protrusions (25) is formed in the top surface of the upper plate (21) in which the osseointegration hole (23a) has not been formed in such a way as to be coupled to the upper spinous process (sp1). The plurality of fixing protrusions (25) is formed in the bottom surface of the lower plate (22) in which the osseointegration hole (23a) has not been formed in such a way as to be coupled to the lower spinous process (sp2).

Referring to FIG. 2, two osseointegration holes (23a) are formed in the upper plate (21), and a plurality of the fixing protrusions (25) is formed in a portion in which the osseointegration hole (23a) has not been formed. Accordingly, the upper plate 21 can be effectively fused with the upper spinous process (sp1). The lower plate (22) has also the same configuration as the upper plate (21).

A side portion of the upper plate (21) that belongs to the upper plate (21) and that corresponds to a portion where the osseointegration hole (23a) is formed further includes an osseointegration hole (23b) formed to penetrate the side portion of the upper plate (21) in such a way as to be connected to the osseointegration hole (23a). A side portion of the lower plate (22) that belongs to the lower plate (22) and that corresponds to a portion where the osseointegration hole (23a) is formed further includes an osseointegration hole (23b) formed to penetrate the side portion of the lower plate (22) in such a way as to be connected to the osseointegration hole (23a).

As shown in FIG. 2, the osseointegration hole (23a) formed to penetrate the upper plate (21) from the top surface of the upper plate (21) to the bottom surface thereof, and the osseointegration hole (23b) formed to penetrate the side of the upper plate (21) are formed to have a structure in which the osseointegration hole (23a) and the osseointegration hole (23b) are connected at the same location of the upper plate (21). Accordingly, when the osseointegration holes (23a) and (23b) are filled with bone powder, the upper plate (21) can be further strongly fused with the upper spinous process (sp1). The same is true of the lower plate (22).

A upper spinous process coupling means (30) is upward rotated by a first rotation rod (31) which is inserted into a through hole (21a) formed to penetrate the side of the upper plate (21) and rotated therein. Next, the upper spinous process coupling means (30) is closely attached to both sides of the upper spinous process sp1 and coupled to the upper spinous process (sp1).

A lower spinous process coupling means (40) is downward rotated by a second rotation rod (41) which is inserted into a through hole (22a) formed to penetrate the side of the lower plate (22) and rotated therein. Next, the lower spinous process coupling means (40) is closely attached to both sides of the lower spinous process sp2 and coupled to the lower spinous process (sp2).

More specifically, upper wings (32 and 33) coupled to a first rotation rod (31) through the first rotation rod (31) are upward rotated. Lower wings (42 and 43) coupled to a second rotation rod (41) through the second rotation rod (41) are downward rotated. The upper wings (32 and 33) and the lower wings (42 and 43) are closely attached and fastened on the first rotation rod (31) and the second rotation rod (41), respectively. Accordingly, a plurality of spinous process fixing spikes (32a and 33a) provided in the upper wings (32 and 33) and a plurality of spinous process fixing spikes (42a and 43a) provided in the lower wings (42 and 43) are embedded in the upper spinous process (sp1) and the lower spinous process (sp2). After the interspinous fusion type implant (10) is inserted between the upper and lower spinous processes (sp1 and sp2), the first rotation rod (31) and the second rotation rod (41) are rotated using a separate tool.

Oblong through holes to which the first rotation rod (31) and the second rotation rod (41) are coupled therethrough are formed in the lower portions of the upper wings (32 and 33) and the lower wings (42 and 43), respectively. The plurality of spinous process fixing spikes (32a, 33a, 42a, and 43a) are formed in the upper portions of the upper wings (32 and 33) and the lower wings (42 and 43), respectively. The outer surfaces of the first rotation rod (31) and the second rotation rod (41) are formed to have shapes capable of rotating the upper wings (32 and 33) and the lower wings (42 and 43) coupled to the first rotation rod (31) and the second rotation rod (41) through the first rotation rod (31) and the second rotation rod (41).

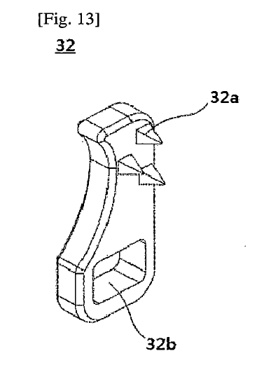

Referring to FIGS. 12 and 13, an oblong through hole (32b) is formed in the lower portion of the upper wing (32), and the plurality of spinous process fixing spike (32a) is provided in the lower portion of the upper wing (32). The remaining wings have the same configuration as the upper wing (32). Screw threads are formed in the outer surface of the first rotation rod (31) and combined with a nut. Both sides of the first rotation rod (31) are cut off in such a manner that the oblong through hole (32b) formed in the upper wing (32) is well combined with the first rotation rod (31). When the first rotation rod (31) is rotated, the upper wing (32) is also rotated.

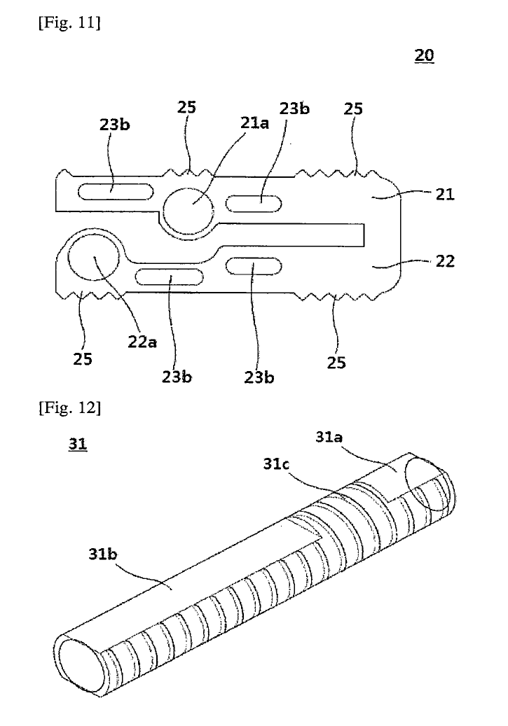

Referring to FIG. 11, the through hole (21a) formed to penetrate the side of the upper plate (21) and the through hole (22a) formed to penetrate the side of the lower plate (22) are formed at mutually dislocated locations. The first rotation rod (31) is provided more on the front side than the second rotation rod (41). By rotating the first rotation rod (31) and the second rotation rod (41) in the same direction using a separate tool, the upper wings (32 and 33) combined with the first rotation rod (31) are upward rotated and the lower wings (42 and 43) combined with the second rotation rod (41) are downward rotated.

The through hole (21a) formed in the upper plate (21) and the through hole (22a) formed in the lower plate (22) are formed at the locations where the through hole (21a) and the through hole (22a) do not overlap. Accordingly, the thickness of the spacer (20) can be prevented from being excessively increased, and the first rotation rod (31) and the second rotation rod (41) can be smoothly rotated without being mutually influenced.

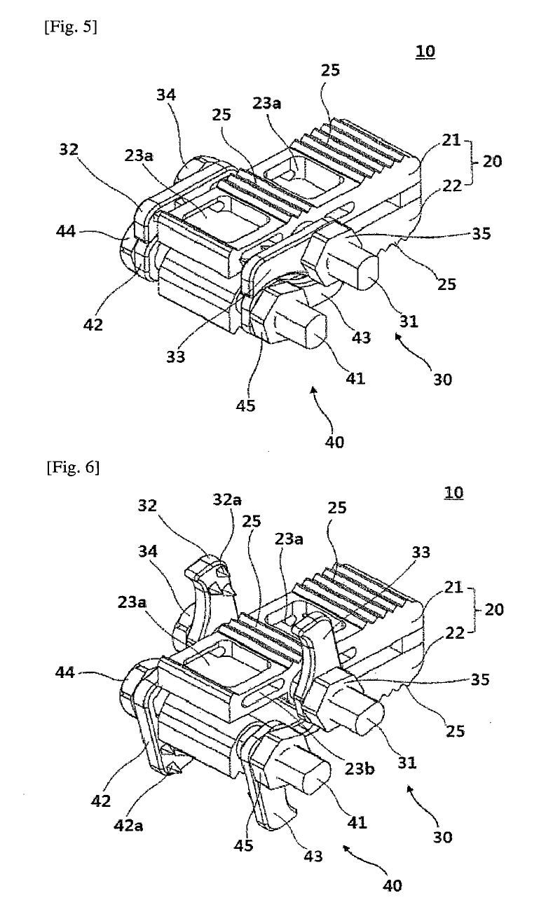

Referring to FIG. 5, when the interspinous fusion type implant is inserted between the adjacent spinous processes (sp1 and sp2), one ends of the upper wings (32 and 33) coupled to the first rotation rod (31) and one ends of the lower wings (42 and 43) coupled to the second rotation rod (41) have been folded in opposite directions.

Referring to FIG. 6, when the first rotation rod (31) and the second rotation rod (41) are rotated using a tool, the folded upper wings (32 and 33) are upward rotated and the folded lower wings (42 and 43) are downward rotated and spread.

Referring to FIGS. 2 to 4, the first rotation rod (31) and the second rotation rod (41) are bolt forms. A bolt head (34, 44) is located on one side of the upper plate (21), the lower plate (22). A bolt body (31, 41) is exposed to the other side of the upper plate (21), the lower plate (22) through the through hole (21a, 22a). A left wing (32) forming an upper wing is coupled to the bolt body (31) exposed between the bolt head (34) and the upper plate (21). A left wing (42) forming a lower wing is coupled to the bolt body (41) exposed between the bolt head (44) and the lower plate (22). A right wing (33) forming an upper wing and a right wing (43) forming a lower wing are coupled to the bolt bodies (31 and 41) exposed to the other side.

Referring to FIG. 12, the outer surface of the bolt body (31) that corresponds to a portion combined with the oblong through hole (32b) formed in the left wing (32, 42) and the right wing (33, 43) includes plurality of faces (31a and 31b) that is flat cut so that the left, right wing is rotated when the bolt body is rotated. The second rotation rod (41) also has the same configuration as the first rotation rod (31).

More specifically, part of the outer surface of the bolt body corresponding to the portion (31a) that belongs to the bolt body (31) and to which the left wing (32, 42) is coupled has been flat cut so that the bolt body can rotate the left wing penetrating the bolt body. Part (31c) of the bolt body extended from the portion to which the left wing (32, 42) is coupled forms a cylindrical body. Part of the outer surface of the bolt body corresponding to the portion (31b) to which the right wing (33, 43) is coupled has been flat cut so that the bolt body can rotate the right wing penetrating the bolt body.

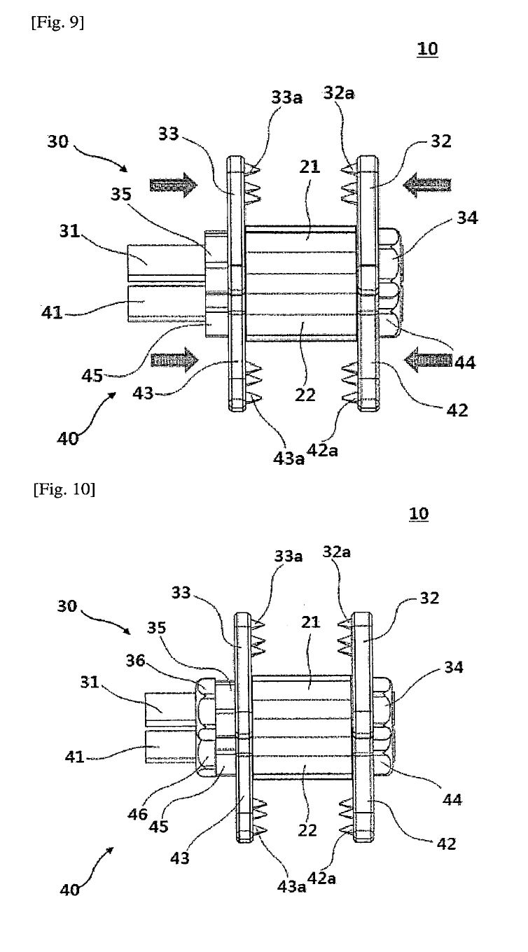

Referring to FIGS. 8 to 10, after the interspinous integrationtype implant (10) is inserted between the adjacent spinous processes (sp1 and sp2), it rotates the first rotation rod (31) and the second rotation rod (41) to upward rotate the upper wings (32 and 33) and to downward rotate the lower wings (42 and 43). In this case, the lower wings (42 and 43) may be first downward rotated earlier than the upper wings (32 and 33).

Fastening nuts (35, 45) are coupled to the outer bolt bodies (31 and 41) of the right wings (33 and 43). The right wing (33) moves in the direction of the left wing (32) along the bolt body (31) by fastening the fastening nut (35) coupled to the first rotation rod (31). When the right wing (33) comes into contact with the side of the upper plate (21) and no longer moves, if the fastening nut (35) continues to be rotated and fastened, the left wing (32) also moves in the direction of the right wing (33) along the bolt body (31), so the left wing (32) is also closely attached to the side of the upper plate (21).

Furthermore, the right wing (43) moves in the direction of the left wing (42) along the bolt body (41) by fastening the fastening nut (45) coupled to the second rotation rod (41). When the right wing (43) comes into contact with the side of the lower plate (22) and no longer moves, if the fastening nut (45) continues to be fastened, the left wing (42) also moves in the direction of the right wing (43) along the bolt body (41), so the left wing (42) is also closely attached to the lower plate (22).

As a result, the left wing (32) and the right wing (33) that form the upper wings are closely attached, and mutually fastened in the direction in which they face each other. The left wing (42) and the right wing (43) that form the lower wings are closely attached and fastened in the direction in which they face each other.

Furthermore, after the left and right wings (32 and 33) are closely attached and fastened by the fastening nut (35), a fixing nut (36) is coupled to the outer bolt body (31) of the fastening nut (35). After the left and right wings (42 and 43) are closely attached and fastened by the fastening nut (45), a fixing nut (46) is coupled to the outer bolt body (41) of the fastening nut (45). By firmly combining and fixing, the fixing nuts (36 and 46), a task for inserting the interspinous integration type implant (10) between the adjacent spinous processes (sp1 and sp2) and coupling the interspinous integration type implant (10) thereto is completed.

In accordance with the present invention, there are advantages in that the space between the upper spinous process and the lower spinous process can be maintained because an elastic force is provided to the upper spinous process and the lower spinous process and the spinous process is not damaged and a phenomenon in which the interspinous fusion type implant escapes between the upper spinous process and the lower spinous process can be prevented because the implant is fused with the upper and lower spinous processes and moved therewith through the osseointegration holes formed in the upper plate and the lower plate.

Furthermore, there is an advantage in that a minimum invasion procedure is possible because the wings are rotated and fastened using a tool after the interspinous fusion type implant is inserted from the sides of the spinous processes.

Furthermore, there is an advantage in that the upper wing and the lower wing are stably coupled to the upper and lower spinous processes because the plurality of spikes included in the upper and lower wings are closely attached to both sides of the upper and lower spinous processes and embedded therein when the upper and lower wings are fastened on both sides of the upper and lower spinous processes after the interspinous fusion type implant is inserted between the adjacent spinous processes.

Furthermore, there is an advantage in that the interspinous fusion type implant can be inserted from the sides of the spinous processes as well as at the back of the spinous processes because the implant can be inserted between the spinous processes in the state in which the upper wing and the lower wing have been folded and thus the wings are not caught in the upper and lower spinous processes.

The present invention is not limited to the specific and preferred embodiment, and a person having ordinary skill in the art to which the present invention pertains may modify the present invention in various ways without departing from the gist of the present invention claimed in the claims. It is evident that such changes fall in the range of the writing of the claims

What is claimed is:

1. An interspinous integrationtype implant inserted between an upper spinous process and a lower spinous process, the implant comprising:
a spacer formed in “ [symbol] ” shape or “U” shape and inserted between spinous processes to provide an elastic force, one ends of an upper plate and a lower plate being connected to the spacer;
upper spinous process coupling means which closely couples the upper plate to an upper spinous process; and
lower spinous process coupling means which closely couples the lower plate to a lower spinous process,
wherein the upper plate comprises one or more osseointegration holes formed to penetrate the upper plate from a top surface of the upper plate to a bottom surface of the upper plate in such a way as to be fused with the upper spinous process and to move, and
the lower plate comprises one or more osseointegration holes formed to penetrate the lower plate from a top surface of the lower plate to a bottom surface of the lower plate in such a way as to be fused with the lower spinous process and to move.

2. The interspinous integrationtype implant of claim 1, wherein:
a plurality of fixing protrusions is formed in the top surface of the upper plate which belongs to the upper plate and in which the osseointegration hole has not been formed and is coupled to the upper spinous process, and
a plurality of fixing protrusions is formed in the bottom surface of the lower plate which belongs to the lower plate and in which the osseointegration hole has not been formed and is coupled to the lower spinous process.

3. The interspinous integrationtype implant of claim 1, wherein:
a side portion of the upper plate corresponding to a portion in which the osseointegration hole is formed further comprises an osseointegration hole formed to penetrate the side portion of the upper plate in such a way as to be connected to the osseointegration hole, and
a side portion of the lower plate corresponding to a portion in which the osseointegration hole is formed further comprises an osseointegration hole formed to penetrate the side portion of the lower plate in such a way as to be connected to the osseointegration hole.

4. The interspinous integrationtype implant of claim 1, wherein:
the upper spinous process coupling means is upward rotated by a first rotation rod inserted into a through hole formed to penetrate a side of the upper plate and rotated with the upper plate, closely attached to both sides of the upper spinous process, and coupled to the upper spinous process, and
the lower spinous process coupling means is downward rotated by a second rotation rod inserted into a through hole formed to penetrate a side of the lower plate and rotated with the lower plate, closely attached to both sides of the lower spinous process, and coupled to the lower spinous process.

5. The interspinous integrationtype implant of claim 4, wherein:
an upper wing coupled to the first rotation rod through the first rotation rod is upward rotated,
a lower wing coupled to the second rotation rod through the second rotation rod is downward rotated, and
the upper wing and the lower wing are closely attached and mutually fastened on the first rotation rod and the second rotation rod, respectively, so a plurality of spinous process fixing spikes provided in the upper wing and a plurality of spinous process fixing spikes provided in the lower wing are embedded in the upper spinous process and the lower spinous process.

6. The interspinous integrationtype implant of claim 5, wherein:
the through hole formed to penetrate the side of the upper plate and the through hole formed to penetrate the side of the lower plate are formed at mutually dislocated locations so that the first rotation rod is provided more on a front side than the second rotation rod, and
the upper wing coupled to the first rotation rod is upward rotated and the lower wing coupled to the second rotation rod is downward rotated by rotating the first rotation rod and the second rotation rod in an identical direction.

7. The interspinous integrationtype implant of claim 5, wherein:
oblong through holes to which the first rotation rod and the second rotation rod are coupled through the oblong through holes are formed on lower sides of the upper wing and the lower wing, respectively,
a plurality of spinous process fixing spikes is provided on an upper side of each of the upper wing and the lower wing, and
outer surfaces of the first rotation rod and the second rotation rod are formed in shapes capable of rotating the upper wing and the lower wing coupled to the first rotation rod and the second rotation rod through the first rotation rod and the second rotation rod.

8. The interspinous integrationtype implant of claim 5, wherein:
the first rotation rod and the second rotation rod have bolt forms,
a bolt head is located on one side of each of the upper plate and the lower plate,
a bolt body is exposed to the other side through the through hole,
a left wing forming the upper wing is coupled to a bolt body exposed between the bolt head and the upper plate,
a left wing forming the lower wing is coupled to a bolt body exposed between the bolt head and the lower plate, and
the right wing forming the upper wing and the right wing forming the lower wing are coupled to the bolt body exposed to the other side.

9. The interspinous integrationtype implant of claim 8, wherein outer surfaces of the bolt bodies corresponding to portions coupled to the oblong through holes formed in the left wing and the right wing comprise a plurality of faces flat cut in such a way as to rotate the left and right wings when the bolt bodies are rotated.

10. The interspinous integrationtype implant of claim 8, wherein:
a fastening nut is coupled to an outer bolt body of the right wing, and
when the fastening nut is fastened, the right wing moves in a direction of the left wing along the bolt body and the left wing moves in a direction of the right wing along the bolt body, so the left wing and the right wing are closely attached and fastened in a direction in which the left wing and the right wing face each other.

11. The interspinous integrationtype implant of claim 10, wherein after the left and right wings are closely attached and fastened by the fastening nut, a fixing nut is coupled to an outer bolt body of the fastening nut.
